(19) United States
(12) Patent Application Publication
(10) Pub. No.: US 2023/0233494 A1
(43) Pub. Date: Jul. 27, 2023

(54) BIOAVAILABLE SUGAR-BASED DICLOFENAC FORMULATIONS

(71) Applicant: APR Applied Pharma Research SA, Balerna (CH)

(72) Inventors: Alberto REINER, Como (IT); Giorgio REINER, Como (IT)

(73) Assignee: APR Applied Pharma Research SA, Balerna (CH)

(21) Appl. No.: 18/001,313

(22) PCT Filed: Jun. 8, 2021
(86) PCT No.: PCT/IB2021/055041

§ 371 Date: Dec. 9, 2022

Related U.S. Application Data
(60) Provisional application No. 63/037,483, filed on Jun. 10, 2020.

(51) Int. Cl. A61K 31/196; A61K 47/10; A61K 47/26; A61K 36/534; A61K 47/02; A61P 29/00

(52) U.S. Cl. CPC A61K 31/196; A61K 47/10; A61K 47/26; A61K 36/534; A61K 47/02; A61P 29/00

(57) ABSTRACT

Ready to use liquid formulations of diclofenac potassium are disclosed which are particularly well suited for packaging in stick-packs.

### FIELD OF THE INVENTION

This invention pertains to a ready to use, liquid, orally administered sugar-based formulations of diclofenac potassium with unexpected bioavailability, chemical stability, and palatability.

### BACKGROUND OF THE INVENTION

Diclofenac potassium ([2-(2,6-dichlorophenyl)amino]benzeneacetate, potassium salt) is a potent NSAID (non-steroidal anti-inflammatory drug) used therapeutically for inflammatory conditions and pain management. The solubility of diclofenac potassium (pKa=3.9) is pH dependent. It is sparingly soluble at acidic pH, and the amount of the active substance dissolved in buffered solutions increases with the increasing pH of the dissolution aqueous medium. The stability of Diclofenac and its salts is well known in the solid state: Diclofenac acid and its salts are in fact characterized by a chemical stability when they are taken in their solid state. When dissolved in water, in contrast, the molecule could be expected to undergo a fast and irreversible oxidative degradation according to the auto-oxidation pathway in FIG. 1.

Diclofenac is sold in various dosage forms, including tablets (Cataflam®), powders for oral solution (Cambia®), gel-caps (Zipsor®), patches (Flector®), and gels (Voltaren®). Other dosage forms are described, inter alia, in WO 2006/133954 (Reiner et al.), WO 1997/044023 (Reiner et al.), and WO 2003/043600 (Reiner et al.). Given its wide spectrum of action and therapeutic benefit, additional dosage forms are needed for convenience of the patient and additional therapeutic uses. These dosage forms should be bioavailable, chemically stable, and palatable to the user.

### SUMMARY OF INVENTION

Therefore, in one embodiment the invention provides a ready to use liquid formulation of diclofenac or a pharmaceutically acceptable salt thereof comprising: (a) 200 weight parts xylitol; (b) from 150 to 1000 weight parts water; and (c) from 0.5 to 10 weight parts diclofenac or a pharmaceutically acceptable salt thereof.

In another embodiment the invention provides a ready to use liquid formulation of diclofenac or a pharmaceutically acceptable salt thereof comprising: (a) 200 weight parts xylitol; (b) from 50 to 500 weight parts water; (c) from 50 to 900 weight parts polyol (preferably sorbitol); and (d) from 0.5 to 10 weight parts diclofenac or a pharmaceutically acceptable salt thereof.

In still another embodiment the invention provides a method of treating a condition selected from pain and migraine in a patient in need thereof comprising administering to said patient a therapeutically effective amount of the formulation of the present invention.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawing, which is incorporated in and constitutes a part of this specification, illustrates several embodiments of the invention and together with the description serves to explain the principles of the invention.

FIG. 1 depicts various auto-oxidation pathways for diclofenac potassium.

### DETAILED DESCRIPTION

### Definitions and Use of Terms

As used in this specification and in the claims which follow, the singular forms “a,” “an” and “the” include plural referents unless the context clearly dictates otherwise.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

“Therapeutically effective amount” means that amount which, when administered to a human for supporting or affecting a metabolic process, or for treating or preventing a disease, is sufficient to cause such treatment or prevention of the disease, or supporting or affecting the metabolic process.

When ranges are given by specifying the lower end of a range separately from the upper end of the range, or specifying particular numerical values, it will be understood that a range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

When used herein the term “about” will compensate for variability allowed for in the pharmaceutical industry and inherent in products in this industry, such as differences in product strength due to manufacturing variation and time-induced product degradation. The term allows for any variation which in the practice of good manufacturing practices would allow the product being evaluated to be considered therapeutically equivalent or bioequivalent in humans to the recited strength of a claimed product.

In the context of the present invention insofar as it relates to any of the disease conditions recited herein, the term “treatment” means to reduce the occurrence of a symptom or condition, or to relieve or alleviate at least one symptom associated with such condition, or to slow or reverse the progression of such condition, or to manage or affect the metabolic processes underlying such condition. Within the meaning of the present invention, the terms also denote to arrest, delay the onset (i.e., the period prior to clinical manifestation of a disease) and/or reduce the risk of developing or worsening a disease.

The phrase “acceptable” as used in connection with compositions of the invention, refers to molecular entities and other ingredients of such compositions that are physiologically tolerable and do not typically produce untoward reactions when administered to a subject (e.g., a mammal such as a human).

### Discussion

In one embodiment the invention provides a ready to use liquid formulation of diclofenac or a pharmaceutically acceptable salt thereof comprising: (a) 200 weight parts xylitol; (b) from 150 to 1000 weight parts water; and (c) from 0.5 to 10 weight parts diclofenac or a pharmaceutically acceptable salt thereof. For ease of reading, the formulations covered by this embodiments will be referred to herein as “the Xylitol Formulations.” As discussed subsequently herein, this terminology does not mean that the formulations are limited to xylitol as the sole polyol, although it will be understood that any of the Xylitol Formulations can contain xylitol as the sole polyol, and that in preferred embodiments the Xylitol Formulations will have xylitol present as the sole polyol.

In another embodiment the invention provides a ready to use liquid formulation of diclofenac or a pharmaceutically acceptable salt thereof comprising: (a) 200 weight parts xylitol; (b) from 50 to 500 weight parts water; (c) from 50 to 900 weight parts polyol (preferably sorbitol); and (d) from 0.5 to 10 weight parts diclofenac or a pharmaceutically acceptable salt thereof. A particularly preferred polyol is sorbitol and an even more preferred sorbitol is non-crystallizing sorbitol, as described in the United States Pharmacopoeia in effect on Dec. 1, 2019. For ease of discussion, formulations covered by this embodiments will be referred to herein as the “the Mixed Polyols Formulations.”

In still another embodiment the invention provides a method of treating a condition selected from pain and migraine in a patient in need thereof comprising administering to said patient a therapeutically effective amount of the formulation of the present invention.

### Alternative Ratios of Weight Parts in Xylitol Formulations

In various subembodiments, the xylitol, water, and diclofenac in the Xylitol Formulations are present in different ratios of weight parts, including:

(a) 200 weight parts xylitol; (b) from 175 to 900 weight parts water; and (c) from 0.75 to 7.5 weight parts diclofenac or a pharmaceutically acceptable salt thereof;

(a) 200 weight parts xylitol; (b) from 100 to 300 weight parts water; and (c) from 0.2 to 2 weight parts diclofenac or a pharmaceutically acceptable salt thereof;

(a) 200 weight parts xylitol; (b) from 150 to 250 weight parts water; and (c) from 0.5 to 1.5 weight parts diclofenac or a pharmaceutically acceptable salt thereof;

(a) 200 weight parts xylitol; (b) from 190 to 210 weight parts water; and (c) from 0.9 to 1.1 weight parts diclofenac or a pharmaceutically acceptable salt thereof;

(a) 200 weight parts xylitol; (b) from 100 to 300 weight parts water; and (c) from 1 to 2.5 weight parts diclofenac or a pharmaceutically acceptable salt thereof;

(a) 200 weight parts xylitol; (b) from 150 to 250 weight parts water; and (c) from 1.2 to 2 weight parts diclofenac or a pharmaceutically acceptable salt thereof;

(a) 200 weight parts xylitol; (b) from 190 to 210 weight parts water; and (c) from 1.6 to 1.8 weight parts diclofenac or a pharmaceutically acceptable salt thereof;

(a) 200 weight parts xylitol; (b) from 450 to 750 weight parts water; and (c) from 2 to 5.5 weight parts diclofenac or a pharmaceutically acceptable salt thereof;

(a) 200 weight parts xylitol; (b) from 525 to 675 weight parts water; and (c) from 2.8 to 4.5 weight parts diclofenac or a pharmaceutically acceptable salt thereof;

(a) 200 weight parts xylitol; (b) from 580 to 620 weight parts water; and (c) from 3.4 to 3.8 weight parts diclofenac or a pharmaceutically acceptable salt thereof;

(a) 200 weight parts xylitol; (b) from 225 to 475 weight parts water; and (c) from 4 to 6.5 weight parts diclofenac or a pharmaceutically acceptable salt thereof;

(a) 200 weight parts xylitol; (b) from 300 to 400 weight parts water; and (c) from 4.5 to 5.7 weight parts diclofenac or a pharmaceutically acceptable salt thereof;

(a) 200 weight parts xylitol; (b) from 335 to 375 weight parts water; and (c) from 4.8 to 5.2 weight parts diclofenac or a pharmaceutically acceptable salt thereof;

(a) 200 weight parts xylitol; (b) from 650 to 1100 weight parts water; and (c) from 4 to 6.5 weight parts diclofenac or a pharmaceutically acceptable salt thereof;

(a) 200 weight parts xylitol; (b) from 750 to 1000 weight parts water; and (c) from 4.5 to 5.7 weight parts diclofenac or a pharmaceutically acceptable salt thereof.

(a) 200 weight parts xylitol; (b) from 820 to 900 weight parts water; and (c) from 4.8 to 5.2 weight parts diclofenac or a pharmaceutically acceptable salt thereof.

In like manner, in various other subembodiments, the xylitol, water, polyol and diclofenac in the Mixed Polyol Formulations are present in different ratios of weight parts, including:

(a) 200 weight parts xylitol; (b) from 260 to 360 weight parts water; (c) from 240 to 320 weight parts polyol (preferably sorbitol); and (d) from 1 to 3 weight parts diclofenac or a pharmaceutically acceptable salt thereof;

(a) 200 weight parts xylitol; (b) from 290 to 330 weight parts water; (c) from 270 to 300 weight parts polyol (preferably sorbitol); and (d) from 1.5 to 2.5 weight parts diclofenac or a pharmaceutically acceptable salt thereof;

(a) 200 weight parts xylitol; (b) from 50 to 150 weight parts water; (c) from 600 to 900 weight parts polyol (preferably sorbitol); and (d) from 1.5 to 3.5 weight parts diclofenac or a pharmaceutically acceptable salt thereof; or

(a) 200 weight parts xylitol; (b) from 60 to 100 weight parts water; (c) from 650 to 800 weight parts polyol (preferably sorbitol); and (d) from 2 to 3 weight parts diclofenac or a pharmaceutically acceptable salt thereof.

### Unit Dosage Forms

The Xylitol Formulations and Mixed Polyol Formulations are preferably present in a unit dosage form comprising a therapeutically effective amount of diclofenac or a pharmaceutically acceptable salt thereof. In various embodiments the therapeutically effective amount comprises about 50 mg of diclofenac or a pharmaceutically acceptable salt thereof. In other embodiments therapeutically effective amount comprises about 50 mg of diclofenac or a pharmaceutically acceptable salt thereof in from about 5 or 8 to about 25 or 50 g (or ml) of said formulation. In other embodiments the therapeutically effective amount comprises about 50 mg of diclofenac or a pharmaceutically acceptable salt thereof in from about 5 or 8 to about 15 g or from about 15 to about 50 g or from about 15 to about 22 g of said formulation. In still other embodiments the therapeutically effective amount comprises about 50 mg of diclofenac or a pharmaceutically acceptable salt thereof in about 20 g of said formulation. The preferred salt of diclofenac in all embodiments is diclofenac potassium.

The unit dosage forms are preferably provided as liquid stick packs that are either consumed as-is, reconstituted in water prior to administration, or consumed as-is followed by the consumption of a liquid chaser. The stick packs are preferably made from one or two sheets of laminate configured to define an interior void sealed around its periphery. The materials used to construct the laminate sheet can be any that are customary in the art, such as polyester, polypropylene, polyethylene and polyethylene terephthalate (PET), provided that the stick pack is sufficiently tear resistant until correctly manipulated. In preferred embodiments the laminate comprises a layer of aluminum foil. Examples of suitable designs for stick packs are described, for example in US 2015/0144518A1 and US20030168375A1. Suitable stick packs can also be purchased from companies such as Unette Corporation (Randolph N.J.), Amcor 360 Packaging Solutions (Melbourne Australia).

In another embodiment the formulation is present in a stick pack marketed as Lamiflex™ 4 by G. Bianchini comprising a trilaminate of polyester, aluminum and polyethylene. In one embodiment the formulation is present in a stick pack comprising a trilaminate of polyester, aluminum and polyethylene, wherein said trilaminate: (a) has a layer thickness of 12/8.5/65 μm, respectively; (a) has a weight of 16.8/22.9/59.9 g/mq, respectively; (c) has micropores in the aluminum layer less than 300/mq.

In another embodiment the formulation is present in a stick pack marketed as PerfecPharm™ P311 by Amcor 360 Packaging Solutions characterized by one or a combination of the following physical properties:

| | Thickness | |
|---|---|---|
| Materials | microns | g/m² |
| BOPET | 12 | 16.9 |
| White PE | 13 | 11.4 |
| Foil | 7 | 19.5 |
| PE | 19 | 17.9 |
| PE Sealant | 32 | 28.2 |

MVTR Barrier (Moisture): <0.02 gms H₂O/m²/24 hours; <0.001 gms H₂O/100 in 2/24 hours (Testing Conditions: 100 F, 90% RH (37.8 C, 90% RH)) (ASTM F-1249)
OTR Barrier (Oxygen): <0.02 cc/m²/24 hours; <0.001 cc/100 in²/24 hours (Testing Conditions: 73 F, 0% RH (23C 0%)) (ASTM D-3985)
Basis Weight: 93.9 g/m² (TM #3001.00/ASTM D-4321)
Gauge: 83.8 microns (TM #3306.00/ASTM D-2103).
   In another embodiment the formulation is present in amber glass vials

### Additional Aspects of the Formulations

The Xylitol Formulations of the present invention can also comprise a polyol in addition to xylitol, preferably selected from ethylene and or propylene glycol; glycerol; erythritol; threitol; arabitol; ribitol; mannitol; sorbitol; galactitol; fucitol; iditol; inositol; volemitol; isomalt; maltitol; lactitol; maltotriitol; maltotetraitol; and polyglycitol. A particularly preferred sorbitol is non-crystallizing sorbitol solution, as described in the United States Pharmacopoeia in effect on Dec. 1, 2019.

Polyols useful in the Mixed Polyol Formulations include, for example, ethylene and or propylene glycol; glycerol; sorbitol erythritol; threitol; arabitol; ribitol; mannitol; galactitol; fucitol; iditol; inositol; volemitol; isomalt; maltitol; lactitol; maltotriitol; maltotetraitol; and polyglycitol. The Mixed Polyol Formulations of the present invention can also comprise a second polyol in addition to xylitol and the first polyol. Preferred second polyols in the Mixed Polyol Formulations are preferably selected from ethylene and or propylene glycol; sorbitol; glycerol; erythritol; threitol; arabitol; ribitol; mannitol; galactitol; fucitol; iditol; inositol; volemitol; isomalt; maltitol; lactitol; maltotriitol; maltotetraitol; and polyglycitol.

Preferred embodiments of the Xylitol Formulation and the Mixed Polyol Formulations do not contain glycerol. Preferred embodiments of the Xylitol Formulation and the Mixed Polyol Formulations also do not contain ethanol.

The Xylitol Formulation and the Mixed Polyol Formulations also preferably comprise an alkalizing agent. Although bicarbonates are preferred alkalizing agents, it will be understood that the formulations can contain any alkalizing agent capable of producing the desired pH (preferably about 7.0 to about 9.5, about 7.5 to about 9.0, or about 8.0 to about 9.0). Such compounds include, by way of example and without limitation, ammonia solution, ammonium carbonate, diethanolamine, monoethanolamine, potassium hydroxide, sodium borate, sodium carbonate, sodium bicarbonate, potassium bicarbonate, sodium hydroxide, triethanolamine, and trolamine and others known to those of ordinary skill in the art. The diclofenac is preferably present in the formulations of the present invention as diclofenac potassium and the alkalizing agent present as potassium bicarbonate, preferably at a weight ratio of about 50:22 (potassium bicarbonte:potassium bicarbonate).

The Xylitol Formulation and the Mixed Polyol Formulations can also comprise additional ingredients selected from the group consisting of thickeners and sweeteners and taste modifying agents. In another embodiment the formulation comprises additional ingredients selected from the group consisting of sucralose, polyvinylpyrrolidone and hydroxyethylcellulose. Suitable taste-masking agents include cellulose hydroxypropyl ethers (HPC); low-substituted hydroxypropyl ethers (L-HPC); cellulose hydroxypropyl methyl ethers (HPMC); methylcellulose polymers; ethylcelluloses (EC) and mixtures thereof; Polyvinyl alcohol (PVA); hydroxyethylcelluloses; carboxymethylcelluloses and salts of carboxymethylcelluloses (CMC); polyvinyl alcohol and polyethylene glycol co-polymers; monoglycerides, triglycerides, polyethylene glycols, modified food starch, acrylic polymers and mixtures of acrylic polymers with cellulose ethers; cellulose acetate phthalate; sepifilms such as mixtures of HPMC and stearic acid, cyclodextrins, and mixtures thereof.

Suitable flavoring agents include acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, neotame, acesulfame potassium, mannitol, talin, xylitol, sucralose, sorbitol, swiss cream, tagatose, tangerine, thaumatin, tutti fruitti, vanilla, walnut, watermelon, wild cherry, wintergreen, xylitol, and mixtures thereof.

The Xylitol Formulation and the Mixed Polyol Formulations can also comprise various buffering agents, stabilizing agents, or antioxidants, including, in particular, EDTA as an antioxidant or chelating agent.

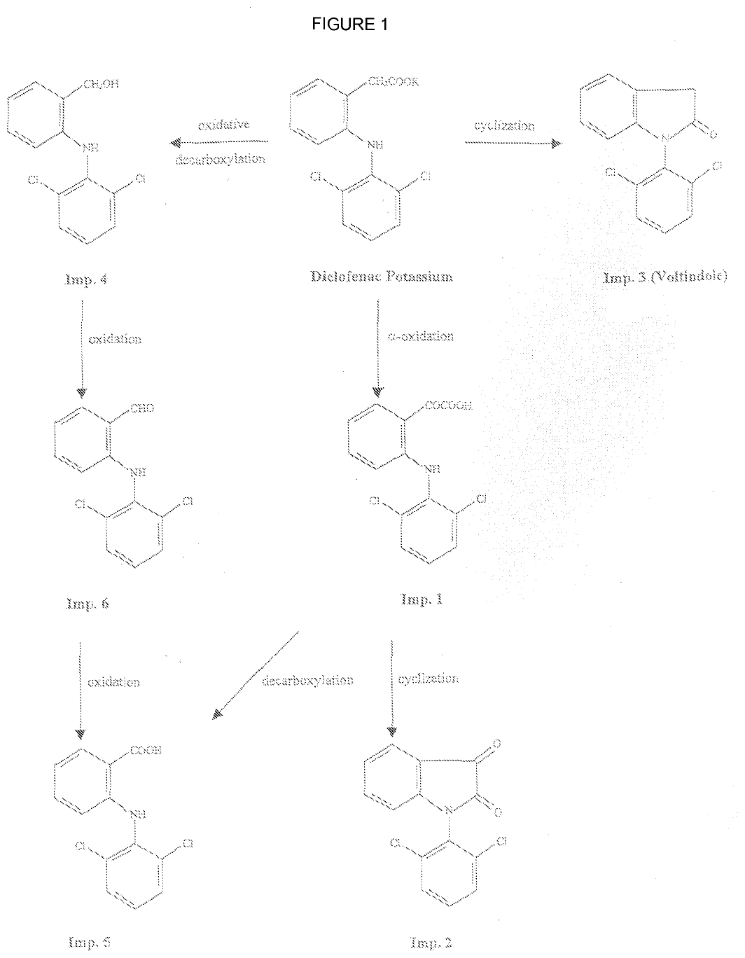

The Xylitol Formulation and the Mixed Polyol Formulations can also be characterized by a density from about 1.02 to about 1.5 g/ml, from about 1.05 to about 1.35 g/ml, or from about 1.1 to about 1.25 g/ml. The Xylitol Formulation and the Mixed Polyol Formulations can also be characterized by a pH of from about 7.0 to about 9.5, or a pH of from about 8.0 to about 9.0. The Xylitol Formulation and the Mixed Polyol Formulations can also be characterized by less than about 1% total impurities, or less than about 1% total impurities after storage at 40° C.±2° C. and 75% RH±5% RH for three or six months. The known impurities are reported in FIG. 1. The known and unknown impurities are not reported in the stability tables of the Examples if their value is lower than 0.1%

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated and are intended to be purely exemplary of the invention and are not intended to limit the scope of what the inventors regard as their invention.

For all the prototypes the Xylisorb™ 300 manufactured by Roquette (Lestrem, France) was used. For all the stability results the reporting threshold for impurities was 0.1%, according to ICH Q3B R2.

### Example 1 Liquid Oral Solution with Diclofenac and Xylitol, with and without Nitrogen (Prototype PFS DK 46-bkT038/122 and Prototype PFS DK 43-bkT038/118)

The following formulations have been prepared to obtain a ready to use liquid solution containing 50 mg of Diclofenac Potassium in 20 g of formula; formulations differ based on the use of nitrogen during the manufacturing.

Ouali/quantitative formulation

| | PFS DK 46 | | PFS DK 43 | |
|---|---|---|---|---|
| | (bkT038/122) | | (bkT038/118) | |
| | mg/stick | | mg/stick | |
| Ingredient | pack | % | pack | % |
| Diclofenac | 50 | 0.250 | 50 | 0.250 |
| potassium | | | | |
| Potassium | 22 | 0.110 | 22 | 0.110 |
| hydrogen | | | | |
| carbonate | | | | |
| Xylitol | 10000 | 50.0 | 10000 | 50.0 |
| Deionized | 9928 | 49.6 | 9928 | 49.6 |
| water | | | | |
| Total (mg) | 20000 | 100.00 | 20000 | 100.00 |

Manufacturing Method: PFS DK 46-bkT038/122

In a glass container transfer the total quantity of water and under stirring add the Xylitol; treat the solution with nitrogen flow for about 30 minutes and wait for the complete dissolution. Add Potassium Bicarbonate and wait for the complete dissolution (about 5 minutes); maintain the system under stirring and under nitrogen flow. Add Diclofenac Potassium and wait at least 2 hours maintaining the system under stirring and under nitrogen flow for all the time. Filter and store the solution in the selected container. Treat the headspace of the container with nitrogen flow before closing the container (amber glass vial).

Manufacturing Method: PFS DK 43-bkT038/118

In a glass container transfer the total quantity of water and, under stirring, add the Xylitol and wait for complete dissolution. Add Potassium Bicarbonate and wait for complete dissolution (about 5 minutes); maintain the system under stirring. Add Diclofenac Potassium and wait at least 2 hours maintaining the system under stirring. Filter and store the solution in the selected container (amber glass vial).

Time zero data

| | PFS DK 46 | PFS DK 43 |
|---|---|---|
| | (bkT038/122) | (bkT038/118) |
| | Time zero | Time zero |
| Appearance of the solution | Complies | Complies |
| pH (on sample, as it is) | 8.42 | 8.59 |
| Diclofenac K Assay (%) | 100.4 | 98.3 |
| Total (known and | — | — |
| unknown) impurities (%) | | |

Stability data PFS DK 46-bkT038/122

| | | Time 3 | Time 3 |
|---|---|---|---|
| | | months | months |
| | | 25° C., | 40° C., |
| PFSDK 46-bkT038/122 | Time zero | 60% RH | 75% RH |
| Appearance of the | Complies | Complies | Slightly |
| solution | | | yellow |
| | | | clear |
| | | | solution |
| pH (on sample, as it is) | 8.42 | 8.93 | 8.80 |
| Diclofenac K Assay (%) | 100.4 | 100.3 | 98.8 |
| Impurity C (4) (%) | — | — | 0.130 |
| Total (known and | — | — | 0.130 |
| unknown) impurities (%) | | | |

Stability data PFS DK 43-bkT038/118

| | | | Time 3 |
|---|---|---|---|
| | | | months |
| PFSDK43- | Tentative | Time | 40° C., |
| bkT038/118 | specifications | zero | 75% RH |
| Appearance of the | Colorless | Complies | Slightly |
| solution | clear solution | | yellow |
| | | | clear |
| | | | solution |
| pH (on sample, as it | To be defined | 8.59 | 8.30 |
| is) | | | |
| Diclofenac K Assay | 95.0-105.0 | 96.2 | 101.0 |
| (%) | | | |
| Impurity C (4) (%) | NMT 0.2 | — | 0.127 |
| Total (known and | NMT 1.0 | — | 0.127 |
| unknown) impurities | | | |
| (%) | | | |

### Example 2 Liquid Oral Solution with Diclofenac, Sorbitol and Xylitol, with and without Nitrogen (Prototype PFS DK 49-bkT038/126 and Prototype PFS DK 44-bkT038/119)

The following formulations have been prepared to obtain a ready to use liquid solution containing 50 mg of Diclofenac Potassium in 20 g of formula; sorbitol and xylitol are mixed for both the prototypes that have the same quali/quantitative formula, but differ based on the use of nitrogen during the manufacturing.

Quali/quantitative formulation

| | PFS DK 49 | | PFS DK 44 | |
|---|---|---|---|---|
| | (bkT038/126) | | (bkT038/119) | |
| | mg/stick | | mg/stick | |
| Ingredient | pack | % | pack | % |
| Diclofenac potassium | 50 | 0.250 | 50 | 0.250 |
| Potassium hydrogen | 22 | 0.110 | 22 | 0.110 |
| carbonate | | | | |
| Non Crystallizing | 7142.8 | 35.7 | 7142.8 | 35.7 |
| Sorbitol | | | | |
| Solution USP 70% | | | | |
| Xylitol | 5000 | 25.0 | 5000 | 25.0 |
| Deionized water | 7785.2 | 38.9 | 7785.2 | 38.9 |
| Total (mg) | 20000 | 100.00 | 20000 | 100.00 |

Manufacturing Method—PFS DK 49-bkT038/126

In a glass container transfer the total quantity of Non crystallizing Sorbitol Solution USP 70% and, under stirring, add the Xylitol and water; treat the solution with nitrogen flow for about 30 minutes. Add Potassium Bicarbonate and wait for the complete dissolution (about 5 minutes); maintain the system under stirring and under nitrogen flow. Add Diclofenac Potassium and wait at least 2 hours maintaining the system under stirring and under nitrogen flow for all the time. Filter and store the solution in the selected container. Treat the headspace of the container with nitrogen flow before close the container (amber glass vial).

Manufacturing Method—PFS DK 44-bkT038/119

In a glass container transfer the total quantity of Non crystallizing Sorbitol Solution 70%, and, under stirring, add the Xylitol and water. Add Potassium Bicarbonate and wait for the complete dissolution (about 5 minutes); maintain the system under stirring. Add Diclofenac Potassium and wait at least 2 hours maintaining the system under stirring. Filter and store the solution in the selected container (amber glass vial).

Time zero data

| | Time zero | Time zero |
|---|---|---|
| | PFS DK 49- | PFS DK 44- |
| | bkT038/126 | bkT038/119 |
| Appearance of the | Complies | Complies |
| solution | | |
| pH (on sample, as it | 8.48 | 8.35 |
| is) | | |
| Diclofenac K Assay | 100.2 | 100.0 |
| (%) | | |
| Impurity C (4) (%) | 0.100 | 0.186 |
| Total (known and | 0.100 | 0.186 |
| unknown) impurities | | |
| (%) | | |

Stability data PFS DK 49-bkT038/119

| | | Time 3 | Time 3 |
|---|---|---|---|
| | | months | months |
| | Time | 25° C., | 40° C., |
| PFS DK 49-bkT038/119 | zero | 60% RH | 75% RH |
| Appearance of the | Complies | Complies | Slightly |
| solution | | | yellow |
| | | | clear |
| | | | solution |
| pH (on sample, as it is) | 8.48 | 8.77 | 8.75 |
| Diclofenac K Assay (%) | 100.2 | 98.6 | 97.6 |
| Impurity C (4) (%) RRT | 0.1 | 0.189 | 0.189 |
| Impurity 5 (%) | — | — | 0.145 |
| UNK 4 (%) RRT = 0.578 | — | — | 0.136 |
| Total (known and | 0.1 | 0.189 | 0.47 |
| unknown) impurities (%) | | | |

Stability data PFS DK 44 - bkT038/126

| | PFS DK 44 - bkT038/126 | | |
|---|---|---|---|
| | | Time 3 | Time 3 |
| | | months | months |
| | | 25° C., | 40° C., |
| | Time zero | 60% RH | 75% RH |
| Appearance of the | Complies | Pale | Slightly |
| solution | | yellow clear | yellow clear |
| | | solution | solution |
| pH (on sample, as it is) | 8.35 | 8.67 | 8.73 |
| Diclofenac K Assay (%) | 100 | 100.1 | 98.5 |
| Impurity C (4) (%) RRT | 0.186 | 0.168 | 0.15 |
| Impurity 5 (%) RRT | — | — | 0.139 |
| UNK 4 (%) RRT = 0.578 | — | — | 0.138 |
| Total (known and | 0.186 | 0.168 | 0.427 |
| unknown) impurities (%) | | | |

### Example 3 Liquid Oral Solution with Diclofenac, Sorbitol and Xylitol, with and without Nitrogen (Prototype PFS DK 48-bkT038/125 and Prototype PFS DK 45-bkT038/121)

The following formulations have been prepared to obtain a ready to use liquid solution containing 50 mg of Diclofenac Potassium in 20 g of formula; sorbitol and xylitol are mixed for both the prototypes that have the same quali/quantitative formula, but differ based on the use of nitrogen during the manufacturing.

| | PFS DK 48 | | PFS DK 45 | |
|---|---|---|---|---|
| | (bkT038/125) | | (bkT038/121) | |
| | mg/stick | | mg/stick | |
| Ingredient | pack | % | pack | % |
| Diclofenac potassium | 50 | 0.250 | 50 | 0.250 |
| Potassium hydrogen | 22 | 0.110 | 22 | 0.110 |
| carbonate | | | | |
| Non Crystallizing Sorbitol | 14285.7 | 71.4 | 7142.8 | 35.7 |
| Solution USP 70% | | | | |
| Xylitol | 4000 | 20.0 | 5000 | 25.0 |
| Deionized water | 1642.3 | 8.2 | 7785.2 | 38.9 |
| Total (mg) | 20000 | 100.0 | 20000 | 100.00 |

For the Manufacturing Methods of-PFS DK 48-bkT038/125 and PFS DK 45-bkT038/120 (in Amber Glass Vials) Refer to Example 2

Time zero data

| | Time zero | Time zero |
|---|---|---|
| | PFS DK 48- | PFS DK 45- |
| | bkT038/125 | bkT038/121 |
| Appearance of the | Complies | Complies |
| solution | | |
| pH (on sample, as it is) | 8.57 | 8.49 |
| Diclofenac K Assay (%) | 101.4 | 101.1 |
| Impurity C (4) (%) | — | 0.150 |
| Total (known and | — | 0.150 |
| unknown) impurities (%) | | |

Stability data PFS DK 48 - bkT038/125

| | PFS DK 48 - bkT038/125 | | |
|---|---|---|---|
| | | Time 3 | Time 3 |
| | | months | months |
| | | 25° C., | 40° C., |
| | Time zero | 60% RH | 75% RH |
| Appearance of the | Complies | Complies | Slightly |
| solution | | | yellow clear |
| | | | solution |
| pH (on sample, as it is) | 8.57 | 8.76 | 8.77 |
| Diclofenac K Assay (%) | 101.4 | 100.2 | 102.1 |
| Impurity C (4) (%) | — | — | 0.104 |
| UNK 4 (%) RRT = 0.578 | — | — | — |
| Total (known and | — | — | 0.104 |
| unknown) impurities (%) | | | |

Stability data PFS DK 45 - bkT038/121

| | PFS DK 45 - bkT038/121 | | |
|---|---|---|---|
| | | Time 3 | Time 3 |
| | | months | months |
| | Time | 25° C., | 40° C., |
| | zero | 60% RH | 75% RH |
| Appearance of the | Complies | Complies | Slightly |
| solution | | | yellow clear |
| | | | solution |
| pH (on sample, as it is) | 8.49 | 8.70 | 8.61 |
| Diclofenac K Assay (%) | 101.1 | 101.0 | 99.6 |
| Impurity C (4) (%) | 0.150 | 0.185 | 0.163 |
| Impurity 5 (%) | — | — | 0.101 |
| Total (known and | 0.150 | 0.185 | 0.264 |
| unknown) impurities (%) | | | |

### Example 4 Liquid Oral Solution with Diclofenac and the 50% of Xylitol, with and without Mint (Prototype PFS DK 161-bkT038/294 and Prototype PFS DK 161-7-bkT038/295)

The following formulations have been prepared to obtain a ready to use liquid solution containing 50 mg of Diclofenac Potassium in 11.8 g of formula; formulations differ for the presence of Mint flavor in the PFS DK 161-7.

Quali/quantitative formulation

| | PFS DK 161 | | PFS DK 161-7 | |
|---|---|---|---|---|
| | (bkT03 8/294) | | (bkT03 8/295) | |
| | mg/stick | | mg/stick | |
| Ingredient | pack | % | pack | % |
| Diclofenac potassium | 50.00 | 0.42 | 50.00 | 0.42 |
| Potassium hydrogen | 22.00 | 0.19 | 22.00 | 0.19 |
| carbonate | | | | |
| Xylitol | 5900.00 | 50.00 | 5900.00 | 50.00 |
| Deionized water | 5828.00 | 49.39 | 5810.30 | 49.24 |
| Mint flavor | — | — | 17.70 | 0.15 |
| Total (mg) | 11800.00 | 100.00 | 11800.00 | 100.00 |

| | | |
|---|---|---|
| Total (ml) | 10.00 | 10.00 |

For the Manufacturing Method—PFS DK 161-bkT038/294 Refer to Example 1 (Stick Pack Laminex 42
Manufacturing Method—PFS DK 161-7-bkT038/295

In a glass container transfer the total quantity of water and, under stirring, add the Xylitol and wait for the complete dissolution. Add Potassium Bicarbonate and wait for the complete dissolution (about 5 minutes); maintain the system under stirring. Add Diclofenac Potassium and wait at least 2 hours maintaining the system under stirring. Add the mint flavor and maintain the system under stirring for 30 minutes. Store the solution in the selected container (Stick Pack Laminex 4).

Time zero data

| | PFS DK 161 | PFS DK 161-7 |
|---|---|---|
| | (bkT038/294) | (bkT038/295) |
| | Time zero | Time zero |
| Appearance of the | Complies | Complies |
| solution | | |
| pH (on sample, as it is) | 8.56 | 8.59 |
| Diclofenac K Assay (%) | 99.3 | 98.9 |
| Total (known and | — | — |
| unknown) impurities (%) | | |

Stability data - Prototype PFS DK 161 bkT038/294

| | | Prototype PFS DK 161 | | |
|---|---|---|---|---|
| | | bkT038/294 | | |
| | | 40° C., | 40° C., | 25° C., |
| | Time | 75% RH | 75% RH | 60% RH |
| | zero | T1 month | T 3 months | |
| Appearance of the | Complies | Complies | Complies | Complies |
| solution | | | | |
| pH (on sample, as it is) | 8.56 | 8.47 | 8.45 | 8.44 |
| Diclofenac K Assay (%) | 99.3 | 98.2 | 98.1 | 99.7 |
| Total (known and | — | — | — | — |
| unknown) impurities (%) | | | | |

Stability data - Prototype PFS DK 161-7 bkT038/295

| | | Prototype PFS DK 161-7 | | |
|---|---|---|---|---|
| | | bkT038/295 | | |
| | | 40° C., | 40° C., | 25° C., |
| | Time | 75% RH | 75% RH | 60% RH |
| | zero | T1 month | T 3 months | |
| Appearance of the | Complies | Complies | Complies | Complies |
| solution | | | | |
| pH (on sample, as it is) | 8.59 | 8.49 | 8.42 | 8.48 |
| Diclofenac K Assay (%) | 98.9 | 101.7 | 101.7 | 102.7 |
| Total (known and | — | — | — | — |
| unknown) impurities (%) | | | | |

### Example 5 Liquid Oral Solution with Diclofenac and the 50% of Xylitol, with and without Mint (Prototype PFS DK 171-bkT038/310 and Prototype PFS DK 171-7-bkT038/311)

The following formulations have been prepared to obtain a ready to use liquid solution containing 50 mg of Diclofenac Potassium in 20 g of formula; formulations differ for the presence of Mint flavor in the PFS DK 171-7.

Quali/quantitative formulation

| | PFS DK 171 | | PFS DK 171-7 | |
|---|---|---|---|---|
| | (bkT038/310) | | (bkT038/311) | |
| | mg/stick | | mg/stick | |
| Ingredient | pack | % | pack | % |
| Diclofenac potassium | 50.00 | 0.25 | 50.00 | 0.25 |
| Potassium hydrogen | 22.00 | 0.11 | 22.00 | 0.11 |
| carbonate | | | | |
| Xylitol | 10000.00 | 50.00 | 10000.00 | 50.00 |
| Deionized water | 9928.00 | 49.64 | 9898.00 | 49.49 |
| Mint flavor | — | — | 30.00 | 0.15 |
| Total (mg) | 20000.00 | 100.00 | 20000.00 | 100.00 |

| | | |
|---|---|---|
| Total (ml) | 16.95 | 16.95 |

Manufacturing Method—PFS DK 171-bkT038/310

Transfer water and xylitol in the equipment and heat up without exceed 40° C. of the solution; mix under vacuum for about 20 minutes combining propeller/contra-propeller (26 rpm/84 rpm); check visually for a complete dissolution and cool the solution to 25-30° C. Add Potassium Hydrogen Bicarbonate and mix under vacuum for about 5-10 minutes combining propeller/contra-propeller (26 rpm/34 rpm). Check visually for a complete dissolution. Add Diclofenac Potassium and mix under vacuum for about 30-60 minutes combining propeller/contra-propeller (26 rpm/34 rpm). Check visually for a complete dissolution. Transfer to the selected container (Stick Pack Lamiflex 4).

Manufacturing Method—PFS DK 171-7-bkT038/311

Transfer water and xylitol in the equipment and heat up without exceed 40° C. of the solution; mix under vacuum for about 20 minutes combining propeller/contra-propeller (26 rpm/84 rpm); check visually for a complete dissolution and cool the solution to 25-30° C. Add Potassium Hydrogen Bicarbonate and mix under vacuum for about 5-10 minutes combining propeller/contra-propeller (26 rpm/34 rpm). Check visually for a complete dissolution. Add Diclofenac Potassium and mix under vacuum for about 30-60 minutes combining propeller/contra-propeller (26 rpm/34 rpm). Check visually for a complete dissolution. Add the mint flavor and stir for 30 minutes. Transfer to the selected container (Stick Pack Lamiflex 4).

Time zero data

| | PFS DK 171 | PFS DK 171-7 |
|---|---|---|
| | (bkT038/310) | (bkT038/311) |
| | Time zero | Time zero |
| Appearance of the | Complies | Complies |
| solution | | |
| pH (on sample, as it is) | 8.26 | 8.30 |
| Diclofenac K Assay (%) | 98.4 | 96.5 |
| Total (known and | — | — |
| unknown) impurities (%) | | |

Stability data - Prototype PFS DK 171bkT038/310

| | | Prototype PFS DK 171 | | | |
|---|---|---|---|---|---|
| | | bkT038/310 | | | |
| | | 40° C., | | 25° C., | 30° C., |
| | Time | 75% RH | | 60% RH | 65% RH |
| | zero | T1 month | T3 months | T6 months | |
| Appearance of the | Complies | Complies | Complies | Complies | Complies |
| solution | | | | | |
| pH (on sample, as it is) | 8.26 | 8.23 | 8.16 | 8.30 | 8.19 |
| Diclofenac K Assay (%) | 98.4 | 96.8 | 95.7 | 98.0 | 96.2 |
| Impurity A (3) (%) | — | — | 0.118 | — | — |
| Total (known and | — | — | 0.118 | — | — |
| unknown) impurities (%) | | | | | |

Stability data - Prototype PFSDK 171-7 bkT038/311

| | | Prototype PFS DK 171-7 | | | |
|---|---|---|---|---|---|
| | | bkT038/311 | | | |
| | | 40° C., | | 25° C., | 30° C., |
| | Time | 75% RH | | 60% RH | 65% RH |
| | zero | T1 month | T3 months | T6 months | |
| Appearance of the | Complies | Complies | Complies | Complies | Complies |
| solution | | | | | |
| pH (on sample, as it is) | 8.30 | 8.19 | 7.90 | 8.22 | 8.21 |
| Diclofenac K Assay (%) | 96.5 | 96.4 | 96.7 | 99.2 | 96.5 |
| Total (known and | — | — | — | — | — |
| unknown) impurities (%) | | | | | |

### Example 6 Liquid Oral Solution with Diclofenac and the 50% of Xylitol, with and without Mint (Prototype PFS DK 172-bkT038/314 and Prototype PFS DK 172-7-bkT038/315)

The following formulations have been prepared to obtain a ready to use liquid solution containing 50 mg of Diclofenac Potassium in 11.8 of formula; formulations differ for the presence of Mint flavor in the PFS DK 172-7. The present formulations represent the big laboratory batches of the PFS DK 161 and PFS DK 161-7.

Quali/quantitative formulation

| | PFS DK 172 | | PFS DK 172-7 | |
|---|---|---|---|---|
| | (bkT038/314) | | (bkT038/315) | |
| | mg/stick | | mg/stick | |
| Ingredient | pack | % | pack | % |
| Diclofenac potassium | 50.00 | 0.42 | 50.00 | 0.42 |
| Potassium hydrogen | 22.00 | 0.19 | 22.00 | 0.19 |
| carbonate | | | | |
| Xylitol | 5900.00 | 50.00 | 5900.00 | 50.00 |
| Deionized water | 5828.00 | 49.39 | 5810.30 | 49.24 |
| Mint flavor | — | — | 17.70 | 0.15 |
| Total (mg) | 11800.00 | 100.00 | 11800.00 | 100.00 |

| | | |
|---|---|---|
| Total (ml) | 10.00 | 10.00 |

For the Manufacturing Method—PFS DK 172-bkT038/314 and PFS DK 172-7-bkT038/315 Refers to Example 5 (Stick Pack Lamiflex 4)

Time zero data

| | PFSDK172 | PFSDK172-7 |
|---|---|---|
| | (bkT038/314) | (bkT038/315) |
| | Time zero | Time zero |
| Appearance of the | Complies | Complies |
| solution | | |
| pH (on sample, as it is) | 8.33 | 8.45 |
| Diclofenac K Assay (%) | 96.8 | 96.7 |
| Total (known and | — | — |
| unknown) impurities (%) | | |

Stability data - Prototype PFS DK172 bkT038/314

| | | Prototype PFS DK 172 | | | | | |
|---|---|---|---|---|---|---|---|
| | | bkT038/314 | | | | | |
| | Time | 40° C., 75% RH | | 30° C., 65% RH | | 25° C., 60% RH | |
| | zero | T1 month | T3 months | T3 months | T6 months | T3 months | T6 months |
| Appearance | Complies | Complies | Slightly | Complies | Complies | Complies | Complies |
| of the | | | yellow | | | | |
| solution | | | clear | | | | |
| | | | solution | | | | |
| pH (on | 8.33 | 8.36 | 8.43 | 8.43 | 8.42 | 8.24 | 8.29 |
| sample, as | | | | | | | |
| it is) | | | | | | | |
| Diclofenac | 96.8 | 97.1 | 96.5 | 95.9 | 96.9 | 97.4 | 99.2 |
| K Assay | | | | | | | |
| (%) | | | | | | | |
| Impurity A | — | — | 0.267 | — | — | — | — |
| (3) (%) | | | | | | | |
| Total | — | — | 0.267 | — | — | — | — |
| (known and | | | | | | | |
| unknown) | | | | | | | |
| impurities | | | | | | | |
| (%) | | | | | | | |

Stability data-Prototype PFSDK 172-7bkT038/315

| | | Prototype PFS DK 172-7bkT038/315 | | | | | |
|---|---|---|---|---|---|---|---|
| | Time | 40° C., 75% RH | | 30° C., 65% RH | | 25° C., 60% RH | |
| | zero | T1 month | T3 months | T3 months | T6 months | T3 months | T6 months |
| Appearance | Complies | Complies | Complies | Complies | Complies | Complies | Complies |
| of the | | | | | | | |
| solution | | | | | | | |
| pH (on | 8.45 | 8.34 | 8.41 | 8.35 | 8.42 | 8..29 | 8.31 |
| sample, | | | | | | | |
| as it is) | | | | | | | |
| Diclofenac | 96.7 | 97.0 | 95.9 | 96.4 | 96.7 | 98.6 | 98.6 |
| K Assay | | | | | | | |
| (%) | | | | | | | |
| Impurity | — | — | 0.155 | — | — | — | — |
| A (3) (%) | | | | | | | |
| Total | — | — | 0.155 | — | — | — | — |
| (known | | | | | | | |
| and | | | | | | | |
| unknown) | | | | | | | |
| impurities | | | | | | | |
| (%) | | | | | | | |

### Example 7 Liquid Oral Solution with Diclofenac and Xylitol, with Sucralose, with and without Mint (Prototype PFS DK 174-bkT038/329 and Prototype PFS DK 174 bkT038/330)

The following formulations have been prepared to obtain a ready to use liquid solution containing 50 mg of Diclofenac Potassium in 11.8 g of formula; formulations differ for the presence of Mint flavor in the PFS DK 174-7.

Quali/quantitative formulation

| | PFS DK 174 | | PFS DK 174-7 | |
|---|---|---|---|---|
| | (bkT038/329) | | (bkT038/330) | |
| | mg/stick | | mg/stick | |
| Ingredient | pack | % | pack | % |
| Diclofenac potassium | 50.00 | 0.42 | 50.00 | 0.42 |
| Potassium hydrogen | 22.00 | 0.19 | 22.00 | 0.19 |
| carbonate | | | | |
| Xylitol | 5900.00 | 50.00 | 5900.00 | 50.00 |
| Deionized water | 5816.20 | 49.29 | 5798.50 | 49.14 |
| Sucralose | 11.80 | 0.10 | 11.80 | 0.10 |
| Mint flavor | — | — | 17.70 | 0.15 |
| Total (mg) | 11800.00 | 100.00 | 11800.00 | 100.00 |

| | | |
|---|---|---|
| Total (ml) | 10.0 | 10.0 |

Manufacturing Method—PFS DK 174-bkT038/329

Transfer water and xylitol in the equipment and heat up without exceed 40° C. of the solution; mix under vacuum for about 20 minutes combining propeller/contra-propeller (26 rpm/84 rpm); check visually for a complete dissolution and cool the solution to 25-30° C. Add Potassium Hydrogen Bicarbonate, Sucralose and mix under vacuum for about 5-10 minutes combining propeller/contra-propeller (26 rpm/34 rpm). Check visually for a complete dissolution. Add Diclofenac Potassium and mix under vacuum for about 30-60 minutes combining propeller/contra-propeller (26 rpm/34 rpm). Check visually for a complete dissolution. Transfer to the selected container (Stick Pack Lamiflex 4).

Manufacturing Method—PFS DK 174-7-bkT038/330

Transfer water and xylitol in the equipment and heat up without exceed 40° C. of the solution; mix under vacuum for about 20 minutes combining propeller/contra-propeller (26 rpm/84 rpm); check visually for a complete dissolution and cool the solution to 25-30° C. Add Potassium Hydrogen Bicarbonate, Sucralose and mix under vacuum for about 5-10 minutes combining propeller/contra-propeller (26 rpm/34 rpm). Check visually for a complete dissolution. Add Diclofenac Potassium and mix under vacuum for about 30-60 minutes combining propeller/contra-propeller (26 rpm/34 rpm). Check visually for a complete dissolution. Add the mint flavor and stir for 30 minutes (Stick Pack Lamiflex 4).

Time zero data

| | PFS DK 174 | PFS DK 174-7 |
|---|---|---|
| | (bkT038/329) | (bkT038/330) |
| | Time zero | Time zero |
| Appearance of the | Complies | Complies |
| solution | | |
| pH (on sample, as it is) | 8.35 | 8.59 |
| Diclofenac K Assay (%) | 99.3 | 97.3 |
| Total (known and | — | — |
| unknown) impurities | | |
| (%) | | |

Stability data-Prototype PFS DK 174bkT038/329

| | | Prototype PFS DK 174bkT038/329 | | | | | | |
|---|---|---|---|---|---|---|---|---|
| | Time | 40° C., 75% RH | | | 30° C., 65% RH | | 25° C., 60% RH | |
| | zero | T1 month | T3 months | T6 months | T3 months | T6 months | T3 months | T6 months |
| Appearance | Complies | Complies | Pale | Pink clear | Complies | Complies | Complies | Complies |
| of the | | | Yellow | solution | | | | |
| solution | | | | | | | | |
| pH (on | 8.35 | 8.24 | 8.05 | 8.19 | 8.19 | 8.24 | 8.22 | 8.26 |
| sample, | | | | | | | | |
| as it is) | | | | | | | | |
| Diclofenac | 99.3 | 97.3 | 94.0 | 94.7 | 96.1 | 96.2 | 95.7 | 95.4 |
| K Assay | | | | | | | | |
| (%) | | | | | | | | |
| Impurity | — | — | 0.227 | 0.745 | — | 0.139 | — | — |
| A (3) (%) | | | | | | | | |
| Impurity | — | — | — | 0.183 | | — | | — |
| 5 (%) | | | | | | | | |
| Total | — | — | 0.227 | 0.928 | — | 0.139 | — | — |
| (known | | | | | | | | |
| and | | | | | | | | |
| unknown) | | | | | | | | |
| impurities | | | | | | | | |
| (%) | | | | | | | | |

Stability data-Prototyve DK 174-7 bkT038/331

| | | Prototype PFS DK 174-7 bkT038/331 | | | | | | |
|---|---|---|---|---|---|---|---|---|
| | Time | 40° C., 75% RH | | | 30° C., 65% RH | | 25 ° C., 60% RH | |
| | zero | T1 month | T3 months | T6 months | T3 months | T6 months | T3 months | T6 months |
| Appearance of the | Complies | Complies | Pale | Pink clear | Complies | Complies | Complies | Complies |
| solution | | | Yellow | solution | | | | |
| pH (on sample, as it | 8.59 | 8.22 | 8.16 | 8.23 | 8.16 | 8.27 | 8.17 | 8.25 |
| is) | | | | | | | | |
| Diclofenac K Assay | 97.3 | 97.0 | 94.7 | 93.8 | 97.5 | 97.5 | 96.1 | 97.8 |
| (%) | | | | | | | | |
| Impurity 2 (%) | — | — | — | — | — | 0.120 | — | — |
| Impurity A (3) (%) | — | — | 0.298 | 0.547 | — | — | — | — |
| Impurity 5 (%) | — | — | — | 0.157 | | — | | — |
| Total (known and | — | — | 0.325 | 0.704 | — | 0.120 | — | — |
| unknown) impurities | | | | | | | | |
| (%) | | | | | | | | |

### Example 8 Liquid Oral Solution with Diclofenac, and the 25% of Xylitol, with and without Mint (Prototype PFS DK 165-bkT038/307 and Prototype PFS DK 165-7-bkT038/317)

The following formulations have been prepared to obtain a ready to use liquid solution containing 50 mg of Diclofenac Potassium in 11.1 g of formula; formulations differ for the presence of Mint flavor in the PFS DK 165-7.

Quali/quantitative formulation

| | PFS DK 165 | | PFS DK 165-7 | |
|---|---|---|---|---|
| | (bkT038/307) | | (bkT038/317) | |
| | mg/stick | | mg/stick | |
| Ingredient | pack | % | pack | % |
| Diclofenac potassium | 50.00 | 0.45 | 50.00 | 0.45 |
| Potassium hydrogen | 22.00 | 0.20 | 22.00 | 0.20 |
| carbonate | | | | |
| Xylitol | 2775.00 | 25.00 | 2775.00 | 25.00 |
| Deionized water | 8253.00 | 74.35 | 8236.35 | 74.20 |
| Mint flavor | — | — | 16.65 | 0.15 |
| Total (mg) | 11100.00 | 100.00 | 11100.00 | 100.00 |

| | | |
|---|---|---|
| Total (ml) | 10.00 | 10.00 |

For the Manufacturing Method—PFS DK 165-bkT038/307 and Manufacturing Method—PFS DK 165-7-bkT038/317 Refers to Example 5 (Stick Pack Lamiflex 4)

Time zero data

| | PFS DK 165 | PFS DK 165-7 |
|---|---|---|
| | (bkT038/307) | (bkT038/317 |
| | Time zero | Time zero |
| Appearance of the | Complies | Complies |
| solution | | |
| pH (on sample, as it | 8.30 | 8.32 |
| is) | | |
| Diclofenac K Assay | 96.5 | 95.4 |
| (%) | | |
| Impurity 1 (%) | — | — |
| Impurity 2 (%) | — | — |
| Impurity A (3) (%) | — | — |
| RRT = 0.60 | | |
| Impurity C (4) (%) | — | — |
| Impurity B (6) (%) | — | — |
| Impurity 5 (%) | — | — |
| Total (known and | — | — |
| unknown) impurities | | |
| (%) | | |

Stability data Prototype PFS DK 165 bkT038/307

| | | Prototype PFS DK 165 bkT038/307 | | | | | | |
|---|---|---|---|---|---|---|---|---|
| | Time | 40° C., 75% RH | | | 30° C., 65% RH | | 25° C., 60% RH | |
| | zero | T1 month | T3 months | T6 months | T3 months | T6 months | T3 months | T6 months |
| Appearance | Complies | Complies | Complies | Pink | Complies | Complies | Complies | Complies |
| of the | | | | clear | | | | |
| solution | | | | solution | | | | |
| pH (on | 8.30 | 8.42 | 8.48 | 8.61 | 8.43 | 8.54 | 8.48 | 8.51 |
| sample, as it | | | | | | | | |
| is) | | | | | | | | |
| Diclofenac K | 96.5 | 95.1 | 93.4 | 92.5 | 96.1 | 95.2 | 97.0 | 95.0 |
| Assay (%) | | | | | | | | |
| Impurity A | — | — | 0.260 | 0.660 | — | 0.131 | — | — |
| (3) (%) | | | | | | | | |
| Impurity 5 | — | — | | | | — | | — |
| (%) | | | | | | | | |
| Total (known | — | — | 0.260 | 0.660 | — | 0.131 | — | — |
| and | | | | | | | | |
| unknown) | | | | | | | | |
| impurities | | | | | | | | |
| (%) | | | | | | | | |

Stability data Prototype PFS DK 165-7 bkT038/317

| | | Prototype PFS DK 165-7 bkT038/317 | | | | | |
|---|---|---|---|---|---|---|---|
| | Time | 40° C., 75% RH | | 30° C., 65% RH | | 25° C., 60% RH | |
| | zero | T1 month | T3 months | T3 months | T6 months | T3 months | T6 months |
| Appearance | Complies | Complies | Complies | Complies | Complies | Complies | Complies |
| of the | | | | | | | |
| solution | | | | | | | |
| pH (on | 8.32 | 8.47 | 8.49 | 8.49 | 8.51 | 8.40 | 8.54 |
| sample, as it | | | | | | | |
| is) | | | | | | | |
| Diclofenac K | 95.4 | 95.9 | 93.7 | 95.2 | 94.8 | 96.2 | 94.7 |
| Assay (%) | | | | | | | |
| Impurity A | — | — | 0.263 | — | 0.140 | — | — |
| (3) (%) | | | | | | | |
| Total (known | — | — | 0.263 | — | 0.140 | — | — |
| and | | | | | | | |
| unknown) | | | | | | | |
| impurities | | | | | | | |
| (%) | | | | | | | |

### Example 9 Liquid Oral Solution with Diclofenac, and the 35% of Xylitol, with and without Mint (Prototype PFS DK 166-bkT038/308 and Prototype PFS DK 166-7-bkT038/318)

The following formulations have been prepared to obtain a ready to use liquid solution containing 50 mg of Diclofenac Potassium in 5.6 g of formula; formulations differ for the presence of Mint flavor in the PFS DK 166-7.

Quali/quantitative formulation

| | PFS DK 166 | | PFS DK 166-7 | |
|---|---|---|---|---|
| | (bkT038/308) | | (bkT038/318) | |
| | mg/stick | | mg/stick | |
| Ingredient | pack | % | pack | % |
| Diclofenac potassium | 50.00 | 0.89 | 50.00 | 0.89 |
| Potassium hydrogen | 22.00 | 0.39 | 22.00 | 0.39 |
| carbonate | | | | |
| Xylitol | 2000.00 | 35.71 | 2000.00 | 35.71 |
| Deionized water | 3528.00 | 63.00 | 3519.60 | 62.85 |
| Mint flavor | — | — | 8.40 | 0.15 |
| Total (mg) | 5600.00 | 100.00 | 5600.00 | 100.00 |

| | | |
|---|---|---|
| Total (ml) | 5.00 | 5.00 |

For the Manufacturing Method—PFS DK 166-bkT038/308 and Manufacturing Method—PFS DK 166-7-bkT038/318 Refers to Example 5 (Stick Pack Lamiflex 4)

Time zero data

| | PFS DK 166 | PFS DK 166-7 |
|---|---|---|
| | (bkT038/308) | (bkT038/318) |
| | Time zero | Time zero |
| Appearance of the | Complies | Complies |
| solution | | |
| pH (on sample, as it | 8.27 | 8.39 |
| is) | | |
| Diclofenac K Assay | 98.9 | 102.9 |
| (%) | | |
| Total (known and | — | — |
| unknown) impurities | | |
| (%) | | |

Stability data - Prototype PFS DK 166 bkT038/308

| | | Prototype PFS DK 166 | |
|---|---|---|---|
| | | bkT038/308 | |
| | Time | 40° C., 75% RH | 40° C., 75% RH |
| | zero | T1month | T3months |
| Appearance of | Complies | Slightly yellow | Clear orange |
| the solution | | clear solution | solution |
| pH (on sample, | 8.27 | 8.44 | 8.47 |
| as it is) | | | |
| Diclofenac K | 98.9 | 97.3 | 96.2 |
| Assay (%) | | | |
| Impurity A (3) | — | 0.185 | 0.412 |
| (%) | | | |
| Total (known | — | 0.185 | 0.412 |
| and unknown) | | | |
| impurities (%) | | | |

Stability data - Prototype PFS DK 166-7 bkT038/318

| | | Prototype PFS DK 166-7 | |
|---|---|---|---|
| | | bkT038/318 | |
| | Time | 40° C., 75% RH | 40° C., 75% RH |
| | zero | T1month | T3months |
| Appearance of | Complies | Complies | Clear orange |
| the solution | | | solution |
| pH (on sample, as it is) | 8.39 | 8.48 | 8.55 |
| Diclofenac K Assay (%) | 102.9 | 97.5 | 95.4 |
| Impurity A (3) (%) | — | 0.134 | 0.278 |
| Total (known and | — | 0.134 | 0.278 |
| unknown) impurities (%) | | | |

### Example 10 Liquid Oral Solution with Diclofenac and the 19% of Xylitol, with and without Mint (Prototype PFS DK 167-bkT038/309 and Prototype PFS DK 167-7-bkT038/319)

The following formulations have been prepared to obtain a ready to use liquid solution containing 50 mg of Diclofenac Potassium in 10.7 g of formula; formulations differ for the presence of Mint flavor in the PFS DK 167-7.

Quali/quantitative formulation

| | PFS DK 167 | | PFS DK 167-7 | |
|---|---|---|---|---|
| | (bkT038/309) | | (bkT038/319) | |
| | mg/stick | | mg/stick | |
| Ingredient | pack | % | pack | % |
| Diclofenac potassium | 50.00 | 0.47 | 50.00 | 0.47 |
| Potassium hydrogen | 22.00 | 0.21 | 22.00 | 0.21 |
| carbonate | | | | |
| Xylitol | 2000.00 | 18.69 | 2000.00 | 18.69 |
| Deionized water | 8628.00 | 80.64 | 8611.95 | 80.49 |
| Mint flavor | — | — | 16.05 | 0.15 |
| Total (mg) | 10700.00 | 100.00 | 10700.00 | 100.00 |

| | | |
|---|---|---|
| Total (ml) | 10.00 | 10.00 |

For the Manufacturing Method—PFS DK 167-bkT038/309 and Manufacturing Method—PFS DK 167-7-bkT038/319 Refers to Example 5 (Stick Pack Lamiflex 4)

Time zero data

| | PFS DK 167 | PFS DK 167-7 |
|---|---|---|
| | (bkT038/309) | (bkT038/319) |
| | Time zero | Time zero |
| Appearance of the | Complies | Complies |
| solution | | |
| pH (on sample, as it | 8.37 | 8.38 |
| is) | | |
| Diclofenac K Assay | 95.5 | 96.6 |
| (%) | | |
| Total (known and | — | — |
| unknown) impurities | | |
| (%) | | |

Stability data Prototype PFS DK 167 bkT038/309

| | | Prototype PFS DK 167 bkT038/309 | | | | | | |
|---|---|---|---|---|---|---|---|---|
| | Time | 40° C., 75% RH | | | 30° C., 65% RH | | 25° C., 60% RH | |
| | zero | T1 month | T3 months | T6 months | T3 months | T6 months | T3 months | T6 months |
| Appearance | Complies | Complies | Complies | Complies | Complies | Complies | Complies | Complies |
| of the | | | | | | | | |
| solution | | | | | | | | |
| pH (on | 8.37 | 8.49 | 8.58 | 8.75 | 8.50 | 8.57 | 8.40 | 8.53 |
| sample, as | | | | | | | | |
| it is) | | | | | | | | |
| Diclofenac K | 95.5 | 96.0 | 94.9 | 93.5 | 95.4 | 96.1 | 96.9 | 98.3 |
| Assay (%) | | | | | | | | |
| Impurity A | — | — | 0.302 | 0.709 | — | 0.140 | — | — |
| (3) (%) | | | | | | | | |
| RRT = 0.60 | | | | | | | | |
| Total | — | — | 0.302 | 0.709 | — | 0.140 | — | — |
| (known and | | | | | | | | |
| unknown) | | | | | | | | |
| impurities | | | | | | | | |
| (%) | | | | | | | | |

Stability data Prototype PFS DK 167-7 bkT038/319

| | | Prototype PFS DK 167-7 bkT038/319 | | | | | |
|---|---|---|---|---|---|---|---|
| | Time | 40° C., 75% RH | | 30° C., 65% RH | | 25° C., 60% RH | |
| | zero | T1 month | T3 months | T1 months | T6 months | T3 months | T6 months |
| Appearance | Complies | Complies | Complies | Complies | Complies | Complies | Complies |
| of the | | | | | | | |
| solution | | | | | | | |
| pH (on | 8.38 | 8.51 | 8.54 | 8.56 | 8.58 | 8.43 | 8.56 |
| sample, as it | | | | | | | |
| is) | | | | | | | |
| Diclofenac K | 96.6 | 96.2 | 95.7 | 95.1 | 95.9 | 96.1 | 98.0 |
| Assay (%) | | | | | | | |
| Impurity A | — | — | 0.197 | — | — | — | — |
| (3) (%) | | | | | | | |
| Impurity C | — | — | — | — | 0.132 | — | — |
| (4) (%) | | | | | | | |
| Total (known | — | — | 0.197 | — | 0.132 | — | — |
| and | | | | | | | |
| unknown) | | | | | | | |
| impurities | | | | | | | |
| (%) | | | | | | | |

### Example 11 Liquid Oral Solution with Diclofenac and the 19% of Xylitol, with and without Mint (Prototype PFS DK 175-bkT038/335 and Prototype PFS DK 175-7-bkT038/336)

The following formulations have been prepared to obtain a ready to use liquid solution containing 50 mg of Diclofenac Potassium in 10.7 g of formula; formulations differ for the presence of Mint flavor in the PFS DK 175-7.

Quali/quantitative formulation

| | PFS DK 175 | | PFS DK 175-7 | |
|---|---|---|---|---|
| | (bkT038/335) | | (bkT038/336) | |
| | mg/stick | | mg/stick | |
| Ingredient | pack | % | pack | % |
| Diclofenac potassium | 50.00 | 0.47 | 50.00 | 0.47 |
| Potassium hydrogen | 22.00 | 0.21 | 22.00 | 0.21 |
| carbonate | | | | |
| Xylitol | 2000.0 | 18.69 | 2000.00 | 18.69 |
| Deionized water | 8617.30 | 80.54 | 8601.25 | 80.39 |
| Sucralose | 10.70 | 0.10 | 10.70 | 0.10 |
| Mint flavor | — | — | 16.05 | 0.15 |
| Total (mg) | 10700.00 | 100.00 | 10700.00 | 100.00 |

| | | |
|---|---|---|
| Total (ml) | 10.00 | 10.00 |

Manufacturing Method—PFS DK 175-bkT038/331

In a glass container transfer the total quantity of water and, under stirring, add the Xylitol and wait for the complete dissolution. Add Potassium Bicarbonate, Sucralose and wait for the complete dissolution (about 5 minutes); maintain the system under stirring. Add Diclofenac Potassium and wait at least 2 hours maintaining the system under stirring; Store the solution in the selected container

Manufacturing Method—PFS DK 175-7-bkT038/332

In a glass container transfer the total quantity of water and, under stirring, add the Xylitol and wait for the complete dissolution. Add Potassium Bicarbonate, Sucralose and wait for the complete dissolution (about 5 minutes); maintain the system under stirring. Add Diclofenac Potassium and wait at least 2 hours maintaining the system under stirring. Add the mint flavor and stir for 30 minutes. Transfer to the selected container

Time zero data

| | PFS DK 175 | PFS DK 175-7 |
|---|---|---|
| | (bkT038/335) | (bkT038/336) |
| | Time zero | Time zero |
| Appearance of the | Complies | Complies |
| solution | | |
| pH (on sample, as it | 8.68 | 8.71 |
| is) | | |
| Diclofenac K Assay | 94.8 | 95.7 |
| (%) | | |
| Total (known and | — | — |
| unknown) impurities | | |
| (%) | | |

### Stability Investigations on the Influence of the Primary Packaging Material

For both the prototypes, the stability was evaluated in two different primary packaging materials in order to investigate their performances
At this purpose two plurilaminate materials have been investigated:_

PerfecPharm™ a plurilaminate manufactured by Perfecseal

Lamiflex 4 a plurilaminate manufactured by Bianchini

The stability data on the flavored prototype (as representative of both the prototypes) are reported.

Stability data Prototype PFS DK 175-7 bkT038/336

| | | | | | Prototype PFS DK 175-7 | |
|---|---|---|---|---|---|---|
| | | Prototype PFS DK 175- 7 | | | bkT038/336 | |
| | | bkT038/336 | | | Stick pack | |
| | | Stick pack (Lamiflex 4) | | | (PerfecPharm ™) | |
| | Time | 40° C., 75% RH | | | 40° C., 75% RH | |
| | zero | T1 month | T3 months | T6 months | T1 month | T3 months |
| Appearance of the | Complies | Complies | Complies | Slightly yellow | Complies | Complies |
| solution | | | | clear solution | | |
| pH (on sample, | 8.71 | 8.46 | 8.27 | 8.19 | 8.53 | 8.36 |
| as it is) | | | | | | |
| Diclofenac K Assay | 95.7 | 95.2 | 92.3 | 93.7 | 95.9 | 92.6 |
| (%) | | | | | | |
| Impurity 1 (%) | — | 0.039 | 0.017 | — | 0.006 | 0.034 |
| Impurity 2 (%) | — | — | — | — | — | — |
| Impurity A (3) (%) | — | — | 0.080 | 0.164 | 0.092 | 0.359 |
| Impurity C (4) (%) | — | — | 0.023 | — | — | 0.026 |
| Impurity B (6) (%) | — | — | — | — | — | — |
| Impurity 5 (%) | — | | 0.063 | 0.141 | — | 0.080 |
| Total (know and | — | 0.039 | 0.183 | 0.305 | 0.098 | 0.499 |
| unknown) impurities | | | | | | |
| (%) | | | | | | |

The stability data collected highlighted the different performances of the two packaging materials; the formulation stored in the PerfecPharm stick packs is characterized by a little bit higher content of impurities at all the storage conditions.
The Lamiflex 4 material seems the most suitable for the packaging of the Diclofenac liquid formulations.

### Example 12 Liquid Oral Solution with Diclofenac and the 50% of Xylitol, without Sucralose, with and without Mint (Prototype PFS DK 176-bkT038/333 and Prototype PFS DK 176-7-bkT038/334)

The following formulations have been prepared to obtain a ready to use liquid solution containing 50 mg of Diclofenac Potassium in 11.8 g of formula; formulations differ for the presence of Mint flavor in the PFS DK 176-7.

Quali/quantitative formulation

| | PFS DK 176 | | PFS DK 176-7 | |
|---|---|---|---|---|
| | (bkT038/333) | | (bkT038/334) | |
| | mg/stick | | mg/stick | |
| Ingredient | pack | % | pack | % |
| Diclofenac potassium | 50.00 | 0.42 | 50.00 | 0.42 |
| Potassium hydrogen | 22.00 | 0.19 | 22.00 | 0.19 |
| carbonate | | | | |
| Xylitol | 5900.0 | 50.0 | 5900.00 | 50.00 |
| Deionized water | 5828.30 | 49.39 | 5810.30 | 49.24 |
| Mint flavor | — | — | 17.70 | 0.15 |
| Total (mg) | 11800.00 | 100.00 | 11800.00 | 100.00 |

| | | |
|---|---|---|
| Total (ml) | 10.00 | 10.00 |

Manufacturing Method—PFS DK 176-bkT038/333

In a glass container transfer the total quantity of water and, under stirring, add the Xylitol and wait for the complete dissolution. Add Potassium Bicarbonate and wait for the complete dissolution (about 5 minutes); maintain the system under stirring. Add Diclofenac Potassium and wait at least 2 hours maintaining the system under stirring; Store the solution in the selected container.

Manufacturing Method—PFS DK 176-7-bkT038/334

In a glass container transfer the total quantity of water and, under stirring, add the Xylitol and wait for the complete dissolution. Add Potassium Bicarbonate and wait for the complete dissolution (about 5 minutes); maintain the system under stirring. Add Diclofenac Potassium and wait at least 2 hours maintaining the system under stirring. Add the mint flavor and stir for 30 minutes. Transfer to the selected container.

Time zero data

| | PFS DK 176 | PFS DK 176-7 |
|---|---|---|
| | (bkT038/333) | (bkT038/334) |
| | Time zero | Time zero |
| Appearance of the | Complies | Complies |
| solution | | |
| pH (on sample, as it | 8.32 | 8.26 |
| is) | | |
| Diclofenac K Assay | 95.7 | 95.2 |
| (%) | | |
| Total (known and | — | — |
| unknown) impurities | | |
| (%) | | |

### Stability Investigations on the Influence of the Primary Packaging Material

Also for these prototypes the stability has been evaluated in the two different primary packaging materials as per the formulations described in Example 11.
The stability data on the flavored prototype (as representative of both the prototypes) are reported.

Stability data Prototype PFS DK 176-7 bkT038/336

| | | Prototype | | Prototype | |
|---|---|---|---|---|---|
| | | PFS DK 176-7 | | PFS DK 176-7 | |
| | | bkT038/334 | | bkT038/334 | |
| | | Stick pack | | Stick pack | |
| | | (Lamiflex 4) | | (PerfecPharm ™) | |
| | Time | 40° C., 75% RH | | 40° C., 75% RH | |
| | zero | T1month | T3months | T1month | T3months |
| Appear- | Complies | Complies | Complies | Complies | Complies |
| ance | | | | | |
| of the | | | | | |
| solution | | | | | |
| pH (on | 8.71 | 8.43 | 8.25 | 8.43 | 8.53 |
| sample, | | | | | |
| as it is) | | | | | |
| Diclofenac | 95.7 | 95.4 | 92.8 | 95.0 | 92.2 |
| K | | | | | |
| Assay (%) | | | | | |
| Impurity 1 | — | 0.003 | 0.016 | 0.008 | 0.018 |
| (%) | | | | | |
| Impurity A | — | 0.027 | 0.035 | 0.081 | 0.279 |
| (3) (%) | | | | | |
| Impurity C | — | — | 0.008 | — | 0.093 |
| (4) (%) | | | | | |
| Impurity B | — | — | — | — | — |
| (6) (%) | | | | | |
| Impurity 5 | — | — | 0.064 | — | — |
| (%) | | | | | |
| Unk 4(%) | — | — | 0.045 | — | — |
| Total | | 0.030 | 0.168 | 0.089 | 0.390 |
| (known | | | | | |
| and | | | | | |
| unknown) | | | | | |
| impurities | | | | | |
| (%) | | | | | |

The stability data collected confirmed the conclusions on the primary packaging material reported in Example 11.
The Lamiflex 4 material seems the most suitable for the packaging of the Diclofenac liquid formulations.

### Example 13 Liquid Oral Solution with Diclofenac and Different Percentage of Xylitol, with and without Sucralose, with and without Mint (Prototypes PFS DK 182-bkT038/346 and Prototype PFS DK 182-7-bkT038/347; Prototypes PFS DK 184-bkT038/340 and Prototype PFS DK 184-7-bkT038/341; Prototypes PFS DK 180-bkT038/342 and Prototype PFS DK 180-7-bkT038/343; Prototypes PFS DK 183-bkT038/348 and Prototype PFS DK 183-7-bkT038/349; Prototypes PFS DK 179-bkT038/340 and Prototype PFS DK 179-7-bkT038/341; Prototypes PFS DK 181-bkT038/344 and Prototype PFS DK 181-7-bkT038/345)

The following formulations have been prepared to confirm the stability of the final prototypes already described in the previous Examples 6-7-8-11-10 in the Lamiflex 4 stick packs

Quali/quantitative formulations (Unflavored)

| | PFS DK 182 | | PFS DK 184 | | PFS DK 180 | | PFS DK 183 | | PFS DK179 | | PFS DK 181 | |
|---|---|---|---|---|---|---|---|---|---|---|---|---|
| | (bkT038/346) | | (bkT038/340) | | (bkT038/342) | | (bkT03 8/348) | | bkT038/340) | | (bkT038/344) | |
| | mg/stick | | mg/stick | | mg/stick | | mg/stick | | mg/stick | | mg/stick | |
| Ingredient | pack | % | pack | % | pack | % | pack | % | pack | % | pack | % |
| Diclofenac | 50.00 | 0.44 | 50.00 | 0.44 | 50.00 | 0.47 | 50.00 | 0.47 | 50.00 | 0.47 | 50.00 | 0.47 |
| potassium | | | | | | | | | | | | |
| Potassium | 22.00 | 0.19 | 22.00 | 0.19 | 22.00 | 0.20 | 22.00 | 0.20 | 22.00 | 0.21 | 22.00 | 0.21 |
| hydrogen | | | | | | | | | | | | |
| carbonate | | | | | | | | | | | | |
| Xylitol | 5899.00 | 49.99 | 5857.10 | 49.64 | 2775.00 | 25.00 | 2775.00 | 25.00 | 2000.0 | 18.69 | 2000.00 | 18.69 |
| Deionized | 5827.00 | 49.38 | 5857.10 | 49.64 | 8251.00 | 74.33 | 8240.30 | 74.24 | 8617.30 | 80.54 | 8628.00 | 80.64 |
| water | | | | | | | | | | | | |
| Sucralose | — | — | 11.80 | 0.10 | — | — | 10.70 | 0.10 | 10.70 | 0.10 | — | — |
| Mint flavor | — | — | — | — | — | — | — | — | — | — | — | — |
| Total (2) | 11.8 | 100.00 | 11.8 | 100.00 | 11.1 | 100.00 | 11.1 | 100.00 | 10.7 | 100.00 | 10.7 | 100.00 |

| | | | | | | |
|---|---|---|---|---|---|---|
| Total (ml) | 10.00 | 10.0 | 10.0 | 10.0 | 10.0 | 10.0 |

Quali/quantitative formulations (Flavored)

| | PFS DK 182-7 | | PFS DK 184-7 | | PFS DK 180-7 | | PFS DK 183-7 | | PFS DK 179-7 | | PFS DK 181-7 | |
|---|---|---|---|---|---|---|---|---|---|---|---|---|
| | (bkT038/347) | | (bkT038/341) | | IbkT038/343) | | (bkT038/349) | | (bkT038/341) | | (bkT038/345 | |
| | mg/stick | | mg/stick | | mg/stick | | mg/stick | | mg/stick | | mg/stick | |
| Ingredient | pack | % | pack | % | pack | % | pack | % | pack | % | pack | % |
| Diclofenac | 50.00 | 0.42 | 50.00 | 0.44 | 50.00 | 0.47 | 50.00 | 0.47 | 50.00 | 0.47 | 50.00 | 0.47 |
| potassium | | | | | | | | | | | | |
| Potassium | 22.00 | 0.19 | 22.00 | 0.19 | 22.00 | 0.20 | 22.00 | 0.20 | 22.00 | 0.21 | 22.00 | 0.21 |
| hydrogen | | | | | | | | | | | | |
| carbonate | | | | | | | | | | | | |
| Xylitol | 5900.00 | 50.00 | 5900.0 | 50.00 | 2775.00 | 25.00 | 2775.00 | 25.00 | 2000.00 | 18.69 | 2000.00 | 18.69 |
| Deionized | 5810.30 | 49.24 | 5798.50 | 49.12 | 8234.35 | 74.18 | 8223.65 | 74.09 | 8601.25 | 80.39 | 8611.95 | 80.49 |
| water | | | | | | | | | | | | |
| Sucralose | — | — | 11.80 | 0.10 | — | — | 10.70 | 0.10 | 10.70 | 0.10 | — | — |
| Mint flavor | 17.70 | 0.15 | 17.70 | 0.15 | 16.65 | 0.15 | 16.65 | 0.15 | 16.05 | 0.15 | 16.05 | 0.15 |
| Total (22) | 11.8 | 100.00 | 11.8 | 100.00 | 11100.00 | 100.00 | 11.1 | 100.00 | 10.7 | 100.00 | 10.7 | 100.00 |

| | | | | | | |
|---|---|---|---|---|---|---|
| Total (ml) | 10.0 | 10.0 | 10.0 | 10.0 | 10.0 | 10.0 |

Stability data Unflavored Prototypes

| | Prototype | | | Prototype | | | Prototype | | |
|---|---|---|---|---|---|---|---|---|---|
| | PFS DK 182 | | | PFS DK 184 | | | PFS DK 180 | | |
| 40° C., | bkT038/346 | | | bkT038/350 | | | bkT038/342 | | |
| 75% RH | T0 | T3m | T6m | T0 | T3m | T6m | T0 | T3m | T6m |
| Appearance | c | c | sycs | c | c | sycs | c | c | sycs |
| of the solution | | | | | | | | | |
| pH (on | 8.36 | 8.13 | 8.09 | 8.44 | 8.22 | 8.08 | 8.55 | 8.15 | 7.49 |
| sample, as | | | | | | | | | |
| it is) | | | | | | | | | |
| Diclofenac | 99.7 | 99.0 | 97.8 | 103.4 | 101.5 | 96.9 | 99.4 | 97.8 | 98.5 |
| K Assay (%) | | | | | | | | | |
| Impurity 1 | — | — | — | — | — | — | — | — | — |
| (%) RRT = | | | | | | | | | |
| 0.38 | | | | | | | | | |
| Impurity 2 | — | — | — | — | — | — | — | — | — |
| (%) RRT = | | | | | | | | | |
| 0.44 | | | | | | | | | |
| Impurity A | — | — | — | — | — | 0.131 | — | — | — |
| (3) (%) | | | | | | | | | |
| RRT = 0.60 | | | | | | | | | |
| Impurity C | — | — | — | — | — | — | — | — | — |
| (4) (%) | | | | | | | | | |
| RRT = 0.82 | | | | | | | | | |
| Impurity B | — | — | — | — | — | — | — | — | — |
| (6) (%) | | | | | | | | | |
| RRT = 1.39 | | | | | | | | | |
| Impurity 5 | — | — | — | — | — | — | — | — | — |
| (%) RRT = | | | | | | | | | |
| 1.12 | | | | | | | | | |
| Unk 1 (%) | — | — | — | — | — | — | — | — | 0.101 |
| RRT = 0.30 | | | | | | | | | |
| Unk 4 (%) | — | — | — | — | — | — | — | 0.164 | 0.378 |
| RRT = 0.74 | | | | | | | | | |
| Unk 10 (%) | — | — | — | — | — | — | — | — | — |
| RRT = 0.86 | | | | | | | | | |
| Total | — | — | — | — | — | 0.131 | — | 0.164 | 0.479 |
| (known and | | | | | | | | | |
| unknown) | | | | | | | | | |
| imp (%) | | | | | | | | | |

| | Prototype | | | Prototype | | | Prototype | |
|---|---|---|---|---|---|---|---|---|
| | PFS DK 183 | | | PFS DK 179 | | | PFS DK181 | |
| 40° C., | bkT038/348 | | | bkT038/340 | | | bkT038/344 | |
| 75% RH | T0 | T3m | T6m | T0 | T3m | T6m | T0 | T3m |
| Appearance | c | c | sycs | c | c | sycs | c | c |
| of the solution | | | | | | | | |
| pH (on | 8.55 | 8.22 | 8.02 | 8.41 | 8.26 | 8.17 | 8.54 | 8.23 |
| sample, as | | | | | | | | |
| it is) | | | | | | | | |
| Diclofenac | 102.7 | 101.3 | 97.4 | 100.2 | 100.2 | 96.7 | 100.2 | 100.3 |
| K Assay (%) | | | | | | | | |
| Impurity 1 | — | — | — | — | — | — | — | — |
| (%) RRT = | | | | | | | | |
| 0.38 | | | | | | | | |
| Impurity 2 | — | — | — | — | — | — | — | — |
| (%) RRT = | | | | | | | | |
| 0.44 | | | | | | | | |
| Impurity A | — | — | — | — | — | 0.160 | — | — |
| (3) (%) | | | | | | | | |
| RRT = 0.60 | | | | | | | | |
| Impurity C | — | — | — | — | — | — | — | — |
| (4) (%) | | | | | | | | |
| RRT = 0.82 | | | | | | | | |
| Impurity B | — | — | — | — | — | — | — | — |
| (6) (%) | | | | | | | | |
| RRT = 1.39 | | | | | | | | |
| Impurity 5 | — | — | — | — | — | — | — | — |
| (%) RRT = | | | | | | | | |
| 1.12 | | | | | | | | |
| Unk 1 (%) | — | — | — | — | — | — | — | — |
| RRT = 0.30 | | | | | | | | |
| Unk 4 (%) | — | — | 0.160 | — | — | — | — | 0.209 |
| RRT = 0.74 | | | | | | | | |
| Unk 10 (%) | — | — | — | — | — | — | — | — |
| RRT = 0.86 | | | | | | | | |
| Total | — | — | 0.160 | — | — | 0.160 | — | 0.209 |
| (known and | | | | | | | | |
| unknown) | | | | | | | | |
| imp (%) | | | | | | | | |

sycs: slightly yellow clear solution
c: complies

Stability data Flavored Prototypes

| | Prototype | | | Prototype | | | Prototype | | |
|---|---|---|---|---|---|---|---|---|---|
| | PFS DK 182-7 | | | PFS DK 184-7 | | | PFS DK 180-7 | | |
| 40° C. | bkT038/347 | | | bkT038/351 | | | bkT038/343 | | |
| 75% HR | T0 | T3m | T6m | T0 | T3m | T6m | T0 | T3m | T6m |
| Appearance | c | c | sycs | c | c | sycs | c | c | sycs |
| of the solution | | | | | | | | | |
| pH (on | 8.50 | 8.38 | 8.09 | 8.49 | 8.14 | 7.99 | 8.54 | 8.16 | 7.85 |
| sample, as | | | | | | | | | |
| it is) | | | | | | | | | |
| Diclofenac | 100.0 | 99.7 | 96.1 | 99.7 | 99.6 | 98.9 | 101.9 | 97.6 | 96.3 |
| K Assay (%) | | | | | | | | | |
| Impurity 1 | — | — | — | — | — | — | — | — | — |
| (%) RRT = | | | | | | | | | |
| 0.38 | | | | | | | | | |
| Impurity 2 | — | — | — | — | — | — | — | — | — |
| (%) RRT = | | | | | | | | | |
| 0.44 | | | | | | | | | |
| Impurity A | — | — | — | — | — | — | — | — | — |
| (3) (%) | | | | | | | | | |
| RRT = 0.60 | | | | | | | | | |
| Impurity C | — | — | — | — | — | — | — | — | — |
| (4) (%) | | | | | | | | | |
| RRT = 0.82 | | | | | | | | | |
| Impurity B | — | — | — | — | — | — | — | — | — |
| (6) (%) | | | | | | | | | |
| RRT = 1.39 | | | | | | | | | |
| Impurity 5 | — | — | — | — | — | — | — | — | 0.181 |
| (%) RRT = | | | | | | | | | |
| 1.12 | | | | | | | | | |
| Unk 4 (%) | — | — | 0.126 | — | — | — | — | 0.148 | 0.324 |
| RRT = 0.74 | | | | | | | | | |
| Unk 10 (%) | — | — | — | — | — | — | — | — | 0.105 |
| RRT = 0.86 | | | | | | | | | |
| Total | — | — | 0.126 | — | — | — | — | 0.148 | 0.612 |
| (known and | | | | | | | | | |
| unknown) | | | | | | | | | |
| imp (%) | | | | | | | | | |

| | Prototype | | | Prototype | | | Prototype | |
|---|---|---|---|---|---|---|---|---|
| | PFS DK 183-7 | | | PFS DK 179-7 | | | PFS DK 181-7 | |
| 40° C. | bkT038/349 | | | bkT038/341 | | | bkT038/345 | |
| 75% HR | T0 | T3m | T6m | T0 | T3m | T6m | T0 | T3m |
| Appearance | c | c | sycs | c | c | sycs | c | c |
| of the solution | | | | | | | | |
| pH (on | 8.48 | 8.03 | 7.74 | 8.45 | 8.30 | 8.12 | 8.55 | 8.23 |
| sample, as | | | | | | | | |
| it is) | | | | | | | | |
| Diclofenac | 99.5 | 101.2 | 97.1 | 100.4 | 97.7 | 95.7 | 100.6 | 99.6 |
| K Assay (%) | | | | | | | | |
| Impurity 1 | — | — | — | — | — | — | — | — |
| (%) RRT = | | | | | | | | |
| 0.38 | | | | | | | | |
| Impurity 2 | — | — | — | — | — | — | — | — |
| (%) RRT = | | | | | | | | |
| 0.44 | | | | | | | | |
| Impurity A | — | — | — | — | — | 0.113 | — | — |
| (3) (%) | | | | | | | | |
| RRT = 0.60 | | | | | | | | |
| Impurity C | — | — | — | — | — | — | — | — |
| (4) (%) | | | | | | | | |
| RRT = 0.82 | | | | | | | | |
| Impurity B | — | — | — | — | — | — | — | — |
| (6) (%) | | | | | | | | |
| RRT = 1.39 | | | | | | | | |
| Impurity 5 | — | — | — | — | — | 0.138 | — | — |
| (%) RRT = | | | | | | | | |
| 1.12 | | | | | | | | |
| Unk 4 (%) | — | 0.130 | 0.227 | — | — | — | — | 0.224 |
| RRT = 0.74 | | | | | | | | |
| Unk 10 (%) | — | — | — | — | — | — | — | — |
| RRT = 0.86 | | | | | | | | |
| Total | — | 130 | 0.227 | — | — | 0.251 | — | 0.224 |
| (known and | | | | | | | | |
| unknown) | | | | | | | | |
| imp (%) | | | | | | | | |

sycs: slightly yellow clear solution
c: complies

All the tested formulations seem stable after 3 and 6 months at 40° C. 75% HR.
The aspect of the solution changed a little bit becoming slightly yellow clear solution
The pH remains quite stable
The Assay of Diclofenac remain in the tentative specifications (95-105%)
All the known impurities remain in the tentative specifications (lower than 0.2%). There is the presence of some unknown impurities (UNK 4 and UNK 10):UNK 4 impurity for the prototypes PFS DK 180 and 180-7 showed a content higher than 0.3%.

The total impurities are lower than 1%

In consideration of the regulatory limits in terms of Xylitol content, the formulations PFS DK 179 and PFS DK 179-7 can be considered the best options because in addition to the stability profile, they are compliant with the FDA guidelines.

### Example 14 Simulated Gastric Fluid Tests

Xylitol based liquid diclofenac prototypes were evaluated in the presence of Simulated Gastric Fluid (SGF) prepared according to USP 42. SGF was prepared by dissolving 2.0 g of sodium chloride and 3.2 g of purified pepsin, derived from porcine stomach mucosa, with an activity of 800 to 2500 units per mg of protein, in 7.0 ml of hydrochloric acid and sufficient water to make 1000 mL. This test solution has a pH of about 1.2. Three different tests were performed:

Test 1—to Mimic the Intake of the Formulations Taken without Water

Dilute a single dose of diclofenac formulation with 45 ml of SGF (37° C.). At the acidic pH value of SGF, diclofenac precipitates. Filter the precipitate, wash it with HCl 0.1N and dry.

### Test 2—to Mimic the Intake of the Formulation Previously Dissolved in a Glass of Water

Dilute a single dose of each diclofenac formulation with 240 ml of drinking water. Add 45 ml of SGF (37° C.). At the acidic pH value of the test solution composed of drinking water and SGF, diclofenac precipitates. Filter the precipitate 5 minutes after the addition of SGF, wash with HCl 0.1N and dry. Centrifuge the filtered solution in order to recover the precipitate eventually passed through the filter, wash with HCl 0.1 N and dry.

### Test 3—to Mimic the Intake of the Formulation Taken Alone, Before a Glass of Water (the Water is Drunk Afterwards)

Dilute a single dose of each diclofenac formulation with 45 ml of SGF (37° C.). After 1 minute, add 240 ml of drinking water. At the acidic pH value of the test solution composed of drinking water and SGF, diclofenac precipitates. Filter the precipitate 5 minutes after the addition of SGF, wash with HCl 0.1N and dry. Centrifuge the filtered solution in order to recover the precipitate eventually passed through the filter, wash with HCl 0.1 N and dry.

Product formulations tested were:
Xylitol based prototypes—Ready to use liquid formulations containing 50 mg/dose of Diclofenac Potassium, PFS DK 172 (bkT038/314) (#1), PFS DK 171 (bkT038/310) (#2), PFS DK 174 (bkT038/329) (#3), PFS DK 165 (bkT038/307) (#4), PFS DK 166 (bkT038/308) (#5), PFS DK 167 (bkT038/309) (#6) and PFS DK 175 (bkT038/331) (#7)
Two reference marketed products, Diclofenac Potassium 50 mg powder for oral solution (#8), and Diclofenac Potassium 50 mg/ml solution for oral solution (#9)

For all reported results, n.a. means there was no (or insufficient) precipitate in the centrifuge test tube to measure, and n.p. means not performed. Total diclofenac recovered means the total diclofenac recovered from the filtered precipitate and the centrifuged precipitate (after drying), and is based either on a 100 mg or 200 mg theoretical recovery.

Xylitol Based Prototypes Test 1 Diclofenac Results

| Formulation | #1 | #2 | #3 | #4 | #5 | #6 | #7 | #8 | #9 |
|---|---|---|---|---|---|---|---|---|---|
| Diclo Assay % of | 0.2 | 0.2 | 0.2 | 0.2 | 0.1 | 0.2 | 0.1 | 0.2 | 0.2 |
| Filtered solution | | | | | | | | | |
| Diclo Assay % of | 97.4 | 95.3 | 95.6 | 89.9 | 94.9 | 96.4 | 96.6 | 94.3 | 98.3 |
| Filtered Precipitate | | | | | | | | | |
| Diclo Assay % of | n.a. | n.a. | n.a. | n.a. | n.a. | n.a. | n.a. | n.p. | n.p. |
| Centrifuged | | | | | | | | | |
| Precipitate | | | | | | | | | |
| Total Diclo (mg) | 183 | 182 | 178 | 170 | 176 | 181 | 180 | n.p. | n.p. |
| recovered | (182 + 1) | (173 + 9) | (169 + 9) | (169 + 1) | (169 + 7) | (180 + 1) | (173 + 7) | | |
| (Theoretical = 200 | | | | | | | | | |
| mg) | | | | | | | | | |

Notes
Instant precipitation was observed for all formulations when SGF was added. Five minutes after filtration, all formulations were clear.

### Conclusion

The xylitol based formulations exhibited similar behavior to the reference marketed products in the Test 1 conditions.

Xylitol Based Prototypes Test 2 Diclofenac Results

| Formulation | #1 | #2 | #3 | #4 | #5 | #6 | #7 | #8 | #9 |
|---|---|---|---|---|---|---|---|---|---|
| Diclo Assay % of | 1.1 | 1.0 | 1.8 | 1.0 | 1.0 | 1.1 | 1.4 | 0.9 | 1.6 |
| Filtered solution | | | | | | | | | |
| Diclo Assay % of | 89.3 | 95.9 | 86.3 | 81.3 | 91.9 | 88.3 | 94.8 | 89.5 | 97.6 |
| Filtered Precipitate | | | | | | | | | |
| Diclo Assay % of | n.a. | n.a. | n.a. | n.a. | n.a. | n.a. | n.a. | 89.3 | 89.1 |
| Centrifuged | | | | | | | | | |
| Precipitate | | | | | | | | | |
| Total Diclo (mg) | 77 | 63 | 64 | 67 | 61 | 62 | 71 | 62 | 55 |
| recovered | (77 + 0) | (62 + 1) | (62 + 2) | (66 + 1) | (61 + 0) | (62 + 0) | (71 + 0) | (12 + 50) | (6 + 49) |
| (Theoretical = 100 | | | | | | | | | |
| mg) | | | | | | | | | |

Notes
Instant precipitation was observed for all formulations when SGF was added. Five minutes after filtration, all formulations were opalescent.

### Conclusion

The xylitol based formulations exhibited similar behavior to the reference marketed products in the Test 2 conditions.

Xylitol Based Prototypes Test 3 Diclofenac Results

| Formulation | #1 | #2 | #3 | #4 | #5 | #6 | #7 | #8 | #9 |
|---|---|---|---|---|---|---|---|---|---|
| Diclo Assay % of | 1.0 | 0.7 | 1.5 | 1.1 | 1.1 | 1.1 | 2.4 | 0.9 | 1.6 |
| Filtered solution | | | | | | | | | |
| Diclo Assay % of | 87.6 | 94.4 | 83.5 | 92.5 | 96.1 | 90.4 | 102.5 | 94.6 | 88.5 |
| Filtered Precipitate | | | | | | | | | |
| Diclo Assay % of | n.a. | n.a. | n.a. | n.a. | n.a. | n.a. | n.a | n.a. | 83.8 |
| Centrifuged | | | | | | | | | |
| Precipitate | | | | | | | | | |
| Total Diclo (mg) | 63 | 57 | 73 | 64 | 70 | 72 | 71 | 67 | 48 |
| recovered | (63 + 0) | (53 + 4) | (71 + 2) | (64 + 0) | (70 + 0) | (72 + 0) | (69 + 2) | (67 + 0) | (29 + 19) |
| (Theoretical = 100 | | | | | | | | | |
| mg) | | | | | | | | | |

Notes
Instant precipitation was observed for all formulations when SGF was added. Five minutes after filtration, formulations 1-8 were clear and formulation 9 was opalescent.

### Conclusions

The xylitol based formulations exhibited the same behavior of the reference marketed products in the Test 3 conditions.

### FINAL CONCLUSIONS

According to the collected data, the xylitol based diclofenac liquid prototypes showed similar behavior to the two reference marketed products in three different methods that simulated three possible ways to take the drug products. The presence of 19-50% xylitol in the formulation (based on the dose weight) in the xylitol prototypes doesn't affect the behavior of diclofenac potassium, which showed the similar precipitation percentage and kinetics observed for the marketed products in the in vitro conditions tested. The similar behavior of the xylitol based formulations and the reference marketed products could be predictive of in vivo behavior similar to the marketed products.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A ready to use liquid formulation of diclofenac or a pharmaceutically acceptable salt thereof comprising:
a) 200 weight parts xylitol;
b) from 150 to 1100 weight parts water; and
c) from 0.5 to 10 weight parts diclofenac or a pharmaceutically acceptable salt thereof.

2. The liquid formulation of claim 1, wherein the formulation comprises:
b) from 175 to 900 weight parts water; and
c) from 0.75 to 7.5 weight parts diclofenac or a pharmaceutically acceptable salt thereof.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The liquid formulation of claim 1, wherein the formulation comprises:
b) from 650 to 1100 weight parts water; and
c) from 4 to 6.5 weight parts diclofenac or a pharmaceutically acceptable salt thereof.

16. The liquid formulation of claim 1, wherein the formulation comprises:
b) from 750 to 1000 weight parts water; and
c) from 4.5 to 5.7 weight parts diclofenac or a pharmaceutically acceptable salt thereof.

17. The liquid formulation of claim 1, wherein the formulation comprises:
b) from 820 to 900 weight parts water; and
c) from 4.8 to 5.2 weight parts diclofenac or a pharmaceutically acceptable salt thereof.

18. The liquid formulation of claim 1, further comprising a flavoring agent selected from the group consisting of mint, sucralose, and a combination thereof.

19. (canceled)

20. (canceled)

21. (canceled)

22. (canceled)

23. (canceled)

24. (canceled)

25. The liquid formulation of claim 1, wherein the formulation has a pH of from 7 to 9.5.

26. The liquid formulation of claim 1, wherein the formulation has a pH of from 7.5 to 9.

27. The liquid formulation of claim 1, further comprising an alkali metal bicarbonate, wherein the formulation has a pH of from 7 to 9.5.

28. The liquid formulation of claim 1, further comprising an alkali metal bicarbonate, wherein the formulation has a pH of from 7.5 to 9.

29. The liquid formulation of claim 1, wherein the formulation does not comprise ethanol and glycerol.

30. (canceled)

31. (canceled)

32. (canceled)

33. (canceled)

34. (canceled)

35. The formulation of claim 1, further comprising additional ingredients selected from the group consisting of thickeners, sweeteners, and taste modifying agents.

36. The formulation of claim 1, further comprising additional ingredients selected from the group consisting of sucralose, polyvinylpyrrolidone, hydroxyethylcellulose, and combinations thereof.

37. The formulation of claim 1, wherein the formulation has a density of from about 1.02 to about 1.5 g/ml.

38. The formulation of claim 1, wherein the formulation has a density of from about 1.05 to about 1.35 g/ml.

39. The formulation of claim 1, wherein the formulation has a density of from about 1.1 to about 1.25 g/ml.

40. A method of treating pain and/or migraine in a patient in need thereof comprising administering to said patient a therapeutically effective amount of the formulation of claim 1.

41. The formulation of claim 1, further comprising potassium hydrogen carbonate.

42. The formulation of claim 1, further comprising a polyol.

43. The formulation of claim 42, wherein the polyol is sorbitol.
